(19) United States
(12) Patent Application Publication
(10) Pub. No.: US 2008/0146968 A1
(43) Pub. Date: Jun. 19, 2008

(54) GAIT ANALYSIS SYSTEM

(76) Inventors: Masuo HANAWAKA, Tokyo (JP); Yota FURUKAWA, Tokyo (JP); Masaharu SAWAMURA, Tokyo (JP); Naoyuki FUJIMOTO, Tokyo (JP)

Correspondence Address: SUGHRUE-265550, 2100 PENNSYLVANIA AVE. NW, WASHINGTON, DC, 20037-3213, US

(21) Appl. No.: 11/955,925

(22) Filed: Dec. 13, 2007

(30) Foreign Application Priority Data
Dec. 14, 2006 (JP) P.2006-336619
Dec. 27, 2006 (JP) P.2006-350744

(51) Int. Cl. A61B 5/00

(52) U.S. Cl. 600/595

(57) ABSTRACT

A gait analysis system has:
a gait sensor which is to be attached to one foot or both feet of a walking person, and which wirelessly outputs detection data of at least one of an acceleration and an angular velocity;
a portable terminal which receives the detection data, and which stores the data for a predetermined time period; and
a gait analyzing apparatus which, based on the detection data obtained from the portable terminal, calculates two- or three-dimensional position information and status information of the foot or feet at an arbitrary time.

This application claims priorities to Japanese Patent Application No. 2006-336619 filed Dec. 14, 2006 and Japanese patent application No. 2006-350744 filed Dec. 27, 2006, in the Japanese Patent Office. The priority applications are incorporated by reference in its entirety.

### TECHNICAL FIELD

The present disclosure relates to a gait analysis system which measures and analyzes a gait state of a walking person to enable a gait pattern of each person to be identifiable, whereby a quantitative index of improvement of the operating efficiency and performance enhancement can be obtained. More particularly, the present disclosure relates to an application to gait pattern analysis in gait training in the rehabilitation field.

### RELATED ART

Patent Reference 1 discloses a technique of gait analysis in which gait data are calculated from results of measurement of a foot pressure distribution obtained by using pressure sensors. FIG. 11 is a panoramic view of the gait analyzing apparatus described in Patent Reference 1.

The invention is characterized in that a person walks on a pressure sensor portion 1101 which is laid in a belt-like manner on a floor, analyzing apparatuses 1102 to 1105 analyze results of measurement of the foot pressure distribution, and a parameter reflecting the level the walking manner is output.

Patent Reference 2 discloses a measuring apparatus in which plural sensors are attached to a person, detection data of the sensors are remotely collected by wireless means, and the body motion is analyzed. FIG. 12 is a diagram of the body motion measuring apparatus described in Patent Reference 2.

The invention is characterized in that detection data of the body motion sensors attached to plural places of a foot are wirelessly sent to a portable receiver to be stored in a storage device of the receiver, the detection data are sent also to an analyzing device, and a result of the analysis is output in various forms.

[Patent Reference 1] Japanese Patent Unexamined Publication No. 11-113884

[Patent Reference 2] Japanese Patent Unexamined Publication No. 10-277015

The gait analyzing techniques of the prior art have the following problems.

(1) In the invention disclosed in Patent Reference 1, the pressure sensor portion for measuring the foot pressure distribution is laid on a floor or the ground, a person walks on the portion, and the gait is analyzed on the basis of the foot pressure distribution during the walking. In this case, the pressure sensor portion must be previously disposed in a place where the person is to walk, and hence a restriction on the installation place is produced.

Since a person walks on the pressure sensor portion, the sensation or consciousness which is different from that of usual walking of the patient is produced, and there is the possibility that the walking operations may result in different manners. The result of walking is restricted to footprints. Therefore, it is impossible to measure and analyze spatial motion of a swing. Depending on the walking distance, plural pressure sensor portions must be prepared.

(2) In the invention disclosed in Patent Reference 2, the plural sensors (an electromyograph, a lower-leg load meter) for analyzing body motion must be attached and fixed to predetermined positions of the subject by dedicated bands. Therefore, the measurement is not simply performed.

There is no means for measuring three-dimensional motion of a gait state, and hence it is impossible to measure and analyze spatial motion of a swing. The wireless means is used in communication between the sensors and the analyzing device which is attached to the waist or the like, and which incorporates a CPU, etc. Therefore, the burden in which the analyzing device is attached to the waist, and which is applied to the patient is not lessened.

The invention is to measure myopotential, lower-leg load, an angle of a joint, and the like of a subject, analyze of the attitude, the number of steps, the heart rate, and the like, and not directed to three-dimensional gait pattern analysis of the subject.

### SUMMARY

Exemplary embodiments of the present invention provide a gait analysis system which can three-dimensionally measure foot motion of the subject, and measure and analyze spatial motion of a swing without applying a burden to the subject.

In order to attain the object, the present invention has the following configurations.

(1) A gait analysis system comprising:

a gait sensor which is to be attached to a foot portion of one foot or both feet of a walking person, and which wirelessly outputs detection data of at least one of an acceleration and an angular velocity;

a wireless communication device which receives the detection data; and

a gait analyzing apparatus which, based on the detection data obtained via the wireless communication device, calculates twos or three-dimensional position information and status information of the foot portion at an arbitrary time.

(2) In a gait analysis system of (1), a start and end of the obtaining of the detection data are determined on the bases of continuation of a suspending state of the foot portion for a constant time period.
(3) In a gait analysis system of (1), detection of a suspending state of the foot portion is determined by satisfying at least one of conditions that a detection value of the angular velocity is not larger than a certain threshold, and that a detection value of the acceleration is not larger than a certain threshold.
(4) In a gait analysis system of (1), the gait sensor comprises at least one of an X-Y-Z direction acceleration sensor and an X-Y-Z direction angular velocity sensor.
(5) In a gait analysis system of (1), the gait sensor is attached to a vicinity of a toe portion of a footwear of the walking person.
(6) In a gait analysis system of (1), the gait sensor is attached to a vicinity of a heel portion of a footwear of the walking person.
(7) In a gait analysis system of (1), the wireless communication device is a wireless access point formed on a network.
(8) In a gait analysis system of (7), the gait analyzing apparatus obtains the detection data from the wireless access point via the network.
(9) In a gait analysis system of (1), the gait analyzing apparatus calculates a relative moving distance by a gait analysis algorithm which processes the detection data, and, based on the relative moving distance, calculates the status information including at least one of data of a gait position, a gait time period, a stride, a gait speed, a ratio of stance, and a ratio of swing.
(10) In a gait analysis system of (1), the gait analyzing apparatus integrates at least one of an angular velocity and an acceleration in each step to calculate an angle, velocity, and moving distance which are two- or three-dimensional.
(11) In a gait analysis system of (1), the gait analyzing apparatus uses at least one of an integration error of the angular velocity, and an integration error of the acceleration ion order to correct a distance error of each step.
(12) In a gait analysis system of (1), the gait analyzing apparatus calculates the position information and the status information in real time with respect to the detection data.
(13) In a gait analysis system of (1), the gait analyzing apparatus does not transmit the detection data of a predetermined time period to the wireless communication device, and holds the detection data in a memory resource of the gait analyzing apparatus.
(14) In a gait analysis system of (1), the gait analyzing apparatus communicates with the gait sensor via the wireless communication device to perform tuning on the gait sensor.

According to the present invention, it is expected to accomplish the following effects.

(1) Gait data can be easily obtained, and hence the system can be readily used at the site of treatment such as a hospital. The gait sensor can be attached to a shoe. Therefore, the walking person is requested only to wear the shoe. Consequently, the subject can freely walk with the feeling which is not different from that in usual walking, and without regard to measurement, with the result that the burden on the subject is very small.
(2) The gait sensor can be mounted in an extremely small size. Therefore, less restriction is imposed on the measurement location. Moreover, the system can be used in a rehabilitation exercise room or the like where many persons exist.
(3) The gait data can be accumulated in the gait analyzing apparatus. Therefore, a change of the gait state can be easily known by comparing the past and present states of the walking person with each other, and the degree of rehabilitation can be quantitatively known.
(4) The measurement is enabled within a radio wave reachable range. Therefore, it is possible to cover a wide walking range (about 100 m) without adding extra apparatuses.
(5) A three-axis acceleration sensor and a three-axis angular velocity (gyroscope) sensor can be incorporated in gait sensor means. When outputs of the sensors are calculation-processed, foot motion in a three-dimensional space can be measured, and a difference in gait pattern can be analyzed vertically, horizontally, and longitudinally.
(6) In application to rehabilitation, particularly, a difference in walking manner due to imperfection content can be three-dimensionally analyzed. Therefore, motion of a foot which is not landing (swing), and which cannot be analyzed by the footprint analysis using a pressure-sensitive mat or the like, can be analyzed.
(7) The number of sensor(s) which can transmit and receive data to and from the wireless access point is not restricted to one. Therefore, even walking motion of plural persons can be simultaneously analyzed.

Further, in order to attain the object, the present invention has the following configurations.

(15) A gait analysis system comprising:

a gait sensor which is to be attached to a foot portion of one foot or both feet of a walking person, and which wirelessly outputs detection data of at least one of an acceleration and an angular velocity;

a portable terminal which receives the detection data, and which stores the data for a predetermined time period; and

a gait analyzing apparatus which, based on the detection data obtained from the portable terminal, calculates two- or three-dimensional position information and status information of the foot portion at an arbitrary time.

(16) In a gait analysis system of (15), the gait sensor comprises at least one of an X-Y-Z direction acceleration sensor and an X-Y-Z direction angular velocity sensor.
(17) In a gait analysis system of (15), the gait sensor is attached to a vicinity of a toe portion of a footwear of the walking person.
(18) In a gait analysis system of (15), the gait sensor is attached to a vicinity of a heel portion of a footwear of the walking person.
(19) In a gait analysis system of (15), the portable terminal is carried by an assistant who is in a vicinity of the walking person.
(20) In a gait analysis system of (15), the gait analyzing apparatus obtains the detection data from the portable terminal via a USB interface.
(21) In a gait analysis system of (15), the gait analyzing apparatus obtains the detection data from the portable terminal via a detachable storing section.
(22) In a gait analysis system of (15), the gait analyzing apparatus calculates a relative moving distance by a gait analysis algorithm which processes the detection data, and, based on the relative moving distance, calculates status information including at least one of data of a gait position, a gait time period, a stride, a gait speed, a ratio of stance, and a ratio of swing.
(23) In a gait analysis system of (15), the gait analyzing apparatus integrates at least one of an angular velocity and an acceleration in each step to calculate an angle, velocity, and moving distance which are two- or three-dimensional.
(24) In a gait analysis system of (15), the gait analyzing apparatus delivers output information to at least one of a doctor, a physical therapist, and the walking person, directly or via a network.

According to the present invention, it is expected to accomplish the following effects.

(1) Gait data can be easily obtained, and hence the system can be readily used at the site of treatment such as a hospital. The gait sensor can be attached to a shoe. Therefore, the walking person is requested only to wear the shoe. Consequently, the subject can freely walk with the feeling which is not different from that in usual walking, and without regard to measurement, with the result that the burden on the subject is very small.
(2) The gait sensor can be mounted in an extremely small size. Therefore, less restriction is imposed on the measurement location. Moreover, the system can be used in a rehabilitation exercise room or the like where many persons exist.
(3) The gait data can be accumulated in the gait analyzing apparatus. Therefore, a change of the gait state can be easily known by comparing the past and present states of the walking person with each other, and the degree of rehabilitation can be quantitatively known.
(4) The assistant can perform measurement in the vicinity of the subject while carrying a small and light portable terminal, and assisting the subject. Since there is no obstacle of wireless communication between the subject and the assistant, stable communication can be ensured.
(5) A three-axis acceleration sensor and a three-axis angular velocity (gyroscope) sensor can be incorporated in gait sensor means. When outputs of the sensors are calculation-processed, foot motion in a three-dimensional space can be measured, and a difference in gait pattern can be analyzed vertically, horizontally, and longitudinally.
(6) In application to rehabilitation, particularly, a difference in walking manner due to imperfection content can be three-dimensionally analyzed. Therefore, motion of a foot which is not landing (swing), and which cannot be analyzed by the footprint analysis using a pressure-sensitive mat or the like, can be analyzed.
(7) The number of sensor(s) which can transmit and receive data to and from the portable terminal is not restricted to one. Therefore, even walking motion of plural persons can be simultaneously measured.

Other features and advantages may be apparent from the following detailed description, the accompanying drawings and the claims.

### BRIEF DESCRIPTION OF THE DRAWINGS

FIG. 1 is a functional block diagram showing the basic configuration of the gait analysis system of the present invention.

FIG. 2 is a functional block diagram showing an example of the configuration of a gait sensor.

FIG. 3 is a functional block diagram showing an example of the configuration of a wireless access point.

FIG. 4 is a functional block diagram showing an example of the configuration of a gait analyzing apparatus.

FIG. 5 is a functional block diagram showing an example of the configuration of a gait data-analyzing portion.

FIG. 6 is a flowchart showing a signal processing procedure of a gait analysis algorithm.

FIG. 7 is a flowchart showing a data processing procedure of a gait analysis application.

FIG. 8 is a functional block diagram showing the basic configuration of the gait analysis system of the present invention.

FIG. 9 is a functional block diagram showing an example of the configuration of a portable terminal.

FIG. 10 is a functional block diagram showing an example of the configuration of a gait analyzing apparatus.

FIG. 11 is a panoramic view of a gait analyzing apparatus described in Patent Reference 1.

FIG. 12 is a diagram of a body motion measuring apparatus described in Patent Reference 2.

### DETAILED DESCRIPTION

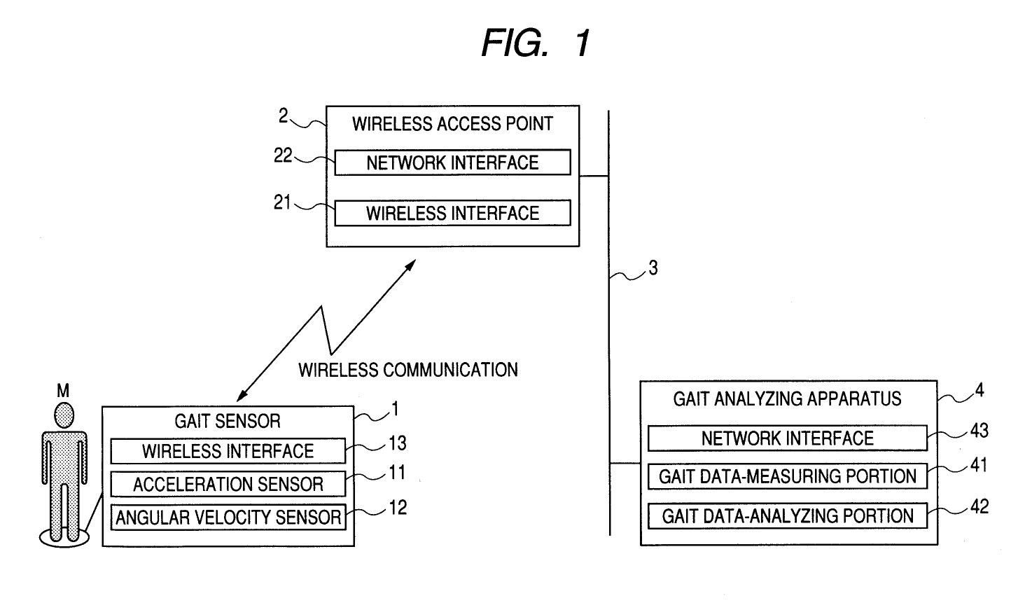

Hereinafter, a first embodiment of the present invention will be described in detail with reference to the accompanying drawings. FIG. 1 is a functional block diagram showing an embodiment of the gait analysis system of the present invention. The gait analysis system of the embodiment is configured by a gait sensor 1, a wireless access point 2, a network 3, and a gait analyzing apparatus 4.

The gait sensor 1 is attached directly to one foot or both feet of a walking person M, or to a footwear (a toe portion or a heel portion), and comprises acceleration sensors 11, angular velocity sensors 12, and a wireless interface 13, so that the gait sensor wirelessly communicates with the wireless access point 2 which is one form of a wireless communication device.

The wireless access point 2 comprises: a wireless interface 21 which communicates with the gait sensor 1; and a network interface 22 which is connected to the network 3. The wireless access point passes gait data collected from the gait sensor 1 to the gait analyzing apparatus 4 via the network 3.

The gait analyzing apparatus 4 comprises a gait data-measuring portion 41 and a gait data-analyzing portion 42, stores the gait data passed from the wireless access point 2, and calculates a relative moving distance by a gait analysis algorithm.

On the basis of the relative moving distance, furthermore, the gait analyzing apparatus 4 executes various analyses by means of a gait analysis application. Items to be analyzed are a gait position, a gait time period, a stride, a gait speed, a ratio of stance, a ratio of swing, etc.

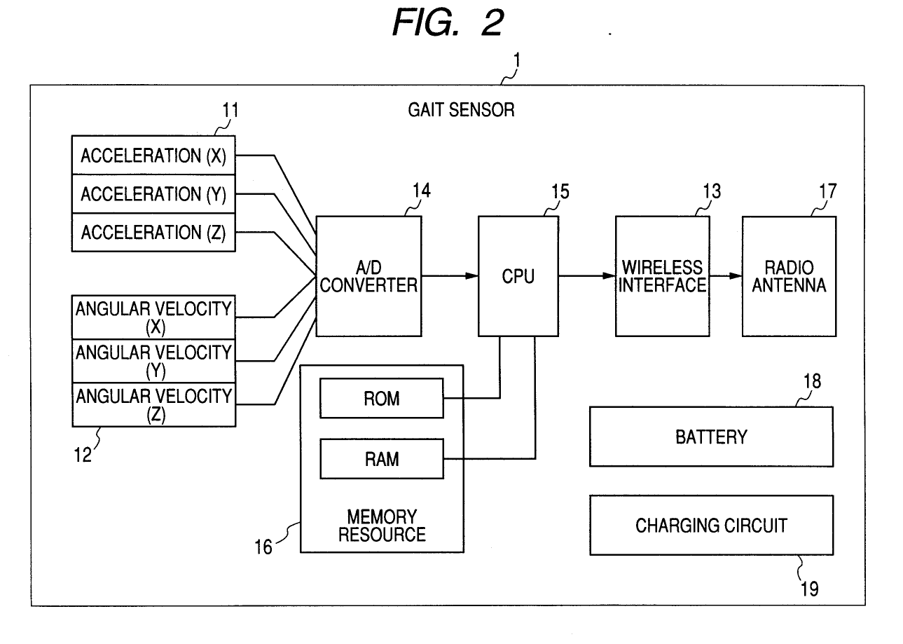

FIG. 2 is a functional block diagram showing an example of the configuration of the gait sensor 1. The gait sensor comprises: three acceleration sensors 11 which detect X-, Y-, Z-axis direction accelerations; three angular velocity sensors 12 which detect X-, Y-, Z-axis direction angular velocities; and an A/D converter 14 which digital-converts detection values of the sensors.

The output of the A/D converter 14 is passed to a CPU 15 to be applied to calculation processes. The CPU 15 has a memory resource 16 such as a ROM and a RPM, so that detection data can be temporarily stored therein.

A result of the calculation of the CPU 15 is transmitted to the wireless access point 2 from a radio antenna 17 via the wireless interface 13. The gait sensor 1 is driven by a rechargeable battery 18, and comprises a charging circuit 19.

The elements constituting the gait sensor 1 can be mounted while they are formed as a very small chip. When the gait sensor is attached directly to one foot or both feet of the walking person, or to a footwear, the walking person can freely walk with the feeling which is not different from that in usual walking, and without regard to measurement, with the result that the burden on the subject is reduced to a very low level.

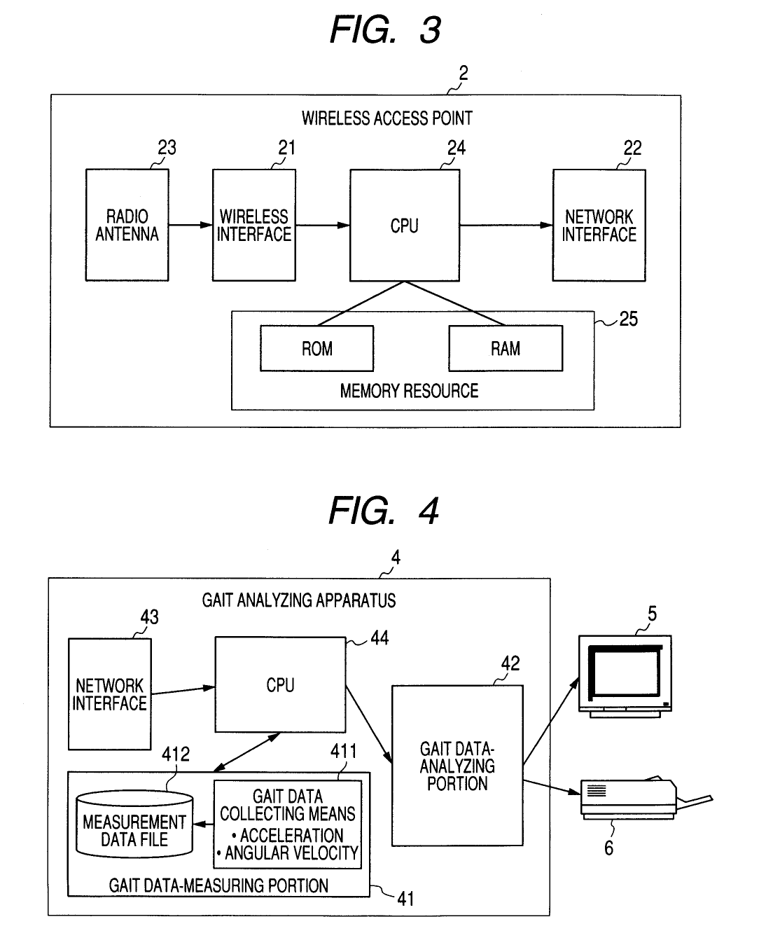

FIG. 3 is a functional block diagram showing an example of the configuration of the wireless access point 2. The data which are received by the wireless interface 21 via an external or internal antenna 23 are processed by a CPU 24, and then supplied to the network 3 via the network interface 22.

The CPU 24 has a memory resource 25 such as a ROM and a RAM, and has a temporary buffer function for the received data. When the amount of the received data accumulated in the buffer reaches a predetermined value, the received data are transmitted to the network 3.

FIG. 4 is a functional block diagram showing an example of the configuration of the gait analyzing apparatus. The data from the wireless access point 2 are passed to a CPU 44 via a network interface 43. Gait data collecting means 411 of the gait data-measuring portion 41 obtains the gait data from the CPU 44, and stores the data in a measurement data file 412 for a predetermined time period. The stored measurement data are acceleration data and angular velocity data.

The CPU 44 reads out the stored data from the measurement data file 412, passes the data to the gait data-analyzing portion 42, and causes the portion to execute various analyzing processes. Results of the analyses are supplied to a displaying device 5 and a printing device 6.

The analysis results can be delivered directly or via the network to an external apparatus or agency, a doctor and physical therapist who are rehabilitation instructors, and the walking person oneself.

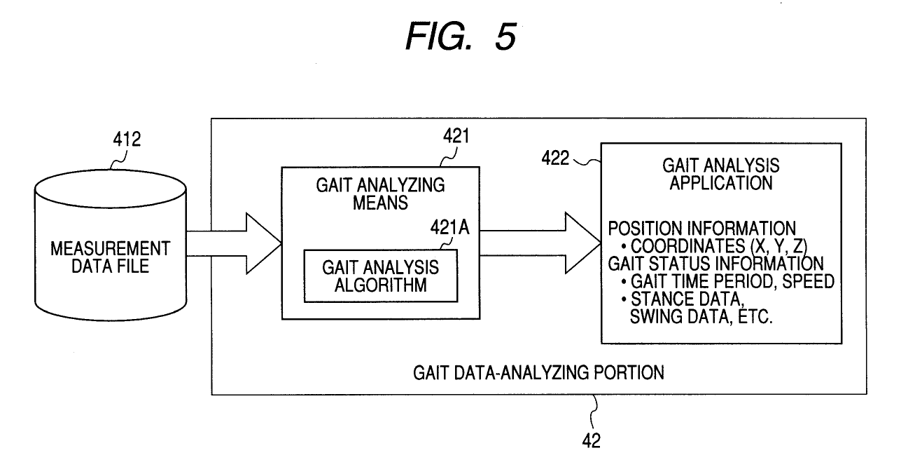

FIG. 5 is a functional block diagram showing an example of the configuration of the gait data-analyzing portion 42. Gait analyzing means 421 retrieves the measurement data stored in the measurement data file 412, passes the data to a gait analysis algorithm 421A to execute various analyses, and supplies results of the analyses to a gait analysis application 422.

Results of the analyses by the gait analysis application 422 include three-dimensional position information of the feet of the walking person, and gait status information of data of a gait time period, a speed, a ratio of stance, and a ratio of swing, etc.

The gait analysis algorithm 421A integrates one time the acceleration data stored in the measurement data file 412, to calculate the velocity. This integration is performed on each of the X, Y, and Z components.

The calculated velocity is integrated one more time to obtain distance data. The angular velocity is similarly integrated to calculate the angle. In this way, the data of the three axes, or the X-, Y-, and Z-axes are calculated, and hence three-dimensional position information can be obtained.

At the same time, in order to calculate the gait status information, the gait analysis algorithm 421A calculates a step segmentation. The step segmentation is determined by, from the acceleration and angular velocity data, calculating a time period when the corresponding foot seems to suspend as a suspending time period, and separating the operating time period from the suspending time period.

The detection of the foot suspending state is determined by satisfying at least one of conditions that the detection value of the angular velocity is not larger than a certain threshold, and that the detection value of the acceleration is not larger than a certain threshold.

From the step segmentation, the gait analysis algorithm 421A calculates status information relating to the step, such as a stance period (a time period when the foot touches the ground), and a swing period (a time period when the foot separates from the ground).

As means for correcting a distance error of each step, at least one of an integration error of the angular velocity, and that of the acceleration can be used. The calculated distance data are converted together with the angle data to coordinate data.

The gait analysis application 422 which obtains the gait data of position information and status information from the gait analyzing means 421 can display the gait data on the displaying device 5 connected to the gait analyzing apparatus 4, and the user can easily know the gait state.

Since past data are stored in the form of files, gait states from the past to the present can be referred, and a change of the gait state can be displayed by a trend graph or the like. If necessary, the change can be output to the printing device 6.

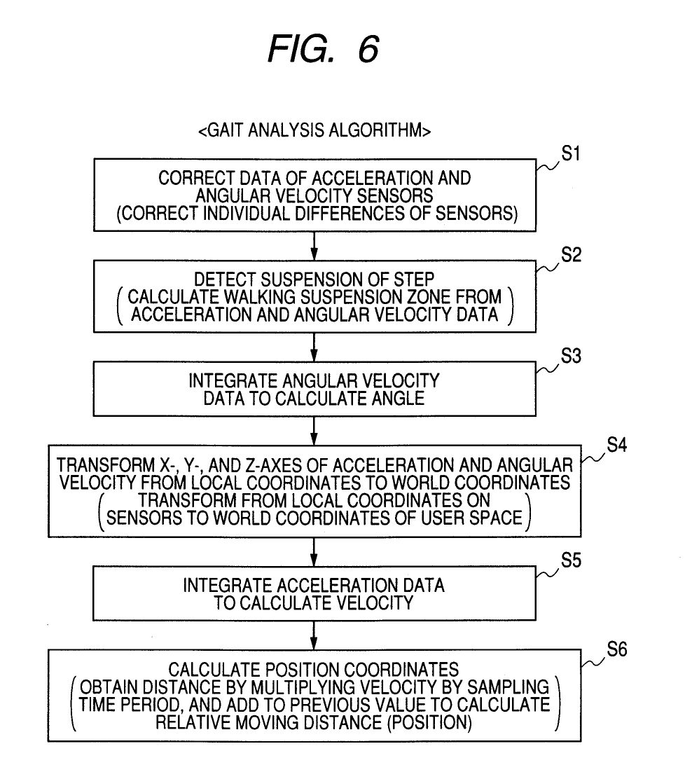

FIG. 6 is a flowchart showing the signal processing procedure of the gait analysis algorithm. In step S1, data based on individual differences of the acceleration sensors and the angular velocity sensors are corrected, and, in step S2, the suspension of the step is detected. Specifically, the walking suspension zone is calculated from the acceleration data and the angular velocity data.

In step S3, the angular velocity data are integrated to calculate the angle. In step S4, the X-, Y-, and Z-axes of the acceleration and the angular velocity are transformed from the local coordinates (the coordinates on the sensors) to the world coordinates (the user space).

In step S5, the acceleration data are integrated to calculate the velocity. In step S6, the position coordinates are calculated. Specifically, the distance is obtained by multiplying the velocity by the sampling time period, and added to the previous value, thereby calculating the relative moving distance (the position).

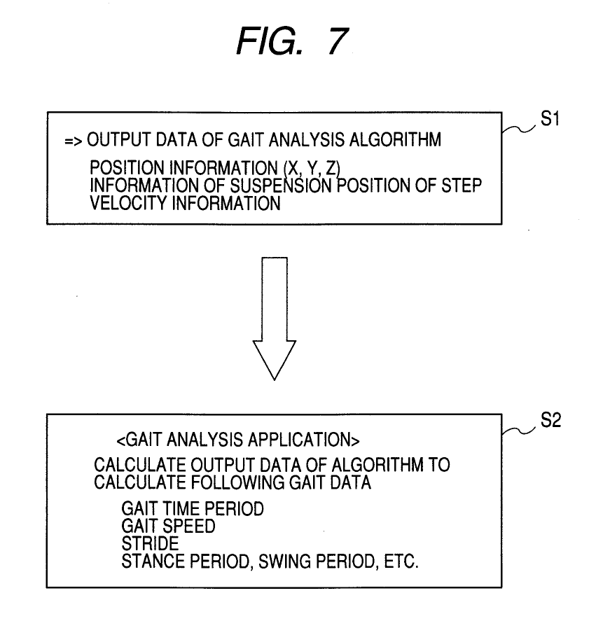

FIG. 7 is a flowchart showing the data processing procedure of the gait analysis application 422. In step S1, as the output data of the gait analysis algorithm 421A, the three-dimensional position information, information of the suspension position of the step, and the velocity information are obtained.

In step S2, the output data obtained in step S1 axe subjected to calculation to calculate status information of a gait such as the gait time period, the gait speed, the stride, the stance period, and the swing period.

Hereinafter, application examples of the present invention will be described. In the embodiment, the example of the signal processing in which the gait analyzing apparatus 4 calculates the position information and the status information in real time with respect to the data has been described. Alternatively, an embodiment in which the gait sensor 1 does not transmit the detection data of a predetermined time period to the wireless communication device, and holds the detection data in its memory resource 16 may be configured.

The gait analyzing apparatus 4 can bi-directionally communicate with the gait sensor 1 via the wireless access point 2. Therefore, the sensitivities and offsets of the acceleration and angular velocity sensors can be tuned to a predetermined value from the side of the gait analyzing apparatus 4.

At the same time when the gait data are accumulated in the files, the data are subjected to calculation in real time, so that the locus of the present gait can be displayed on the screen. Therefore, it is possible to see the present status of the walking person.

Since the gait locus data can be obtained, it is possible to know motion of a person in a production site or the like. Also the motion of a patient in a hospital can be known.

The connection between the wireless access point 2 and the gait analyzing apparatus 4 can be realized also by means other than the network 3, for example, a USB or a wireless LAN.

The embodiment in which the gait analyzing apparatus 4 executes the calculation process on the data has been described. Alternatively, the calculation process may be performed in the sensor 1, and calculation data may be displayed and accumulated in the portable terminal or the like, thereby allowing the gait analysis system to be established by simpler apparatuses.

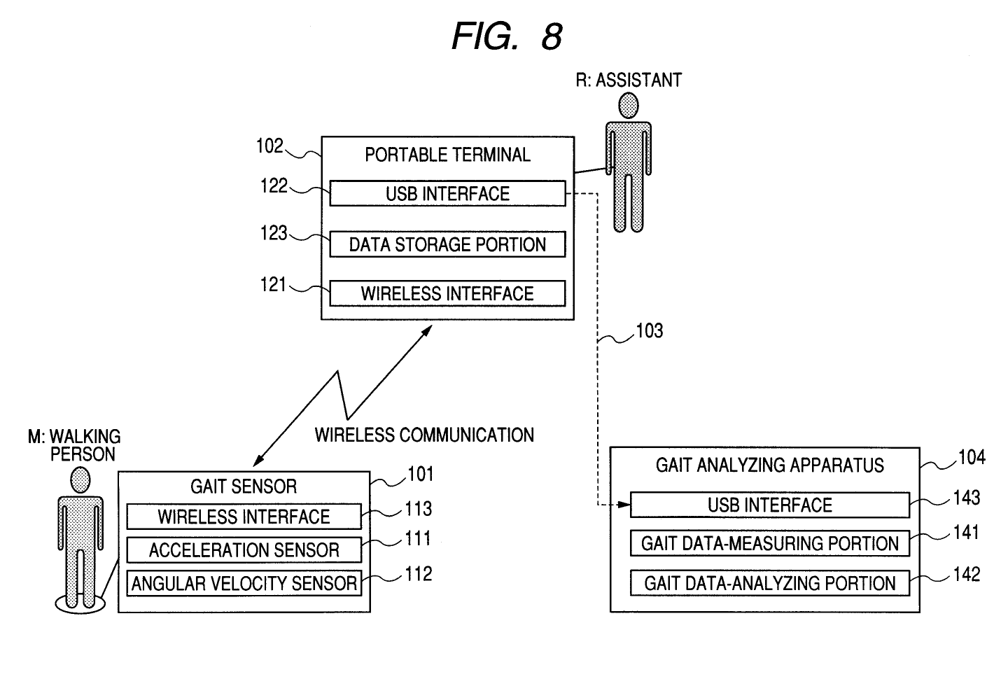

Hereinafter, a second embodiment of the present invention will be described in detail with reference to the accompanying drawings. FIG. 8 is a functional block diagram showing an embodiment of the gait analysis system of the present invention. The gait analysis system of the embodiment is configured by a gait sensor 101, a portable terminal 102, a USB cable 103, and a gait analyzing apparatus 104.

The gait sensor 101 is attached directly to one foot or both feet of a walking person M, or to a footwear (a toe portion or a heel portion), and comprises acceleration sensors 111, angular velocity sensors 112, and a wireless interface 113, so that the gait sensor wirelessly communicates with the portable terminal 102 carried by an assistant R.

The portable terminal 102 comprises: a wireless interface 121 which communicates with the gait sensor 101; a USB interface 122 which is connected to the USB cable 103; and a data storage portion 123. The portable terminal stores gait data collected from the gait sensor 101 for a predetermined time period, and passes the data to the gait analyzing apparatus 104 via the USB cable 103.

The gait analyzing apparatus 104 comprises a gait data-measuring portion 141 and a gait data-analyzing portion 142, stores the gait data passed from the portable terminal 102, and calculates a relative moving distance by a gait analysis algorithm.

On the basis of the relative moving distance, furthermore, the gait analyzing apparatus 104 executes various analyses by means of a gait analysis application. Items to be analyzed are a gait position, a gait time period, a stride, a gait speed, a ratio of stance, a ratio of swing, etc.

The gait sensor 101 has, for example, the same configuration of the first embodiment as shown in FIG. 2. The gait sensor 101 of the second embodiment will be explained by using FIG. 2. The gait sensor comprises: three acceleration sensors 11 (the acceleration sensor 111 of FIG. 8) which detect X-, Y-, Z-axis direction accelerations; three angular velocity sensors 12 (the angular velocity 112 of FIG. 8) which detect X-, Y-, Z-axis direction angular velocities; and an A/D converter 14 which digital-converts detection values of the sensors.

The output of the A/D converter 14 is passed to a CPU 15 to be applied to calculation processes such as primary filtering. The CPU 15 has a memory resource 16 such as a ROM and a RAM, so that detection data can be temporarily stored therein.

A result of the calculation of the CPU 15 is transmitted to the portable terminal 102 from a radio antenna 17 via the wireless interface 113. The gait sensor 101 is driven by a rechargeable battery 18, and comprises a charging circuit 19.

The elements constituting the gait sensor 101 can be mounted while they are formed as a very small chip. When the gait sensor is attached directly to one foot or both feet of the walking person, or to a footwear, the walking person can freely walk with the feeling which is not different from that in usual walking, and without regard to measurement, with the result that the burden on the subject is reduced to a very low level.

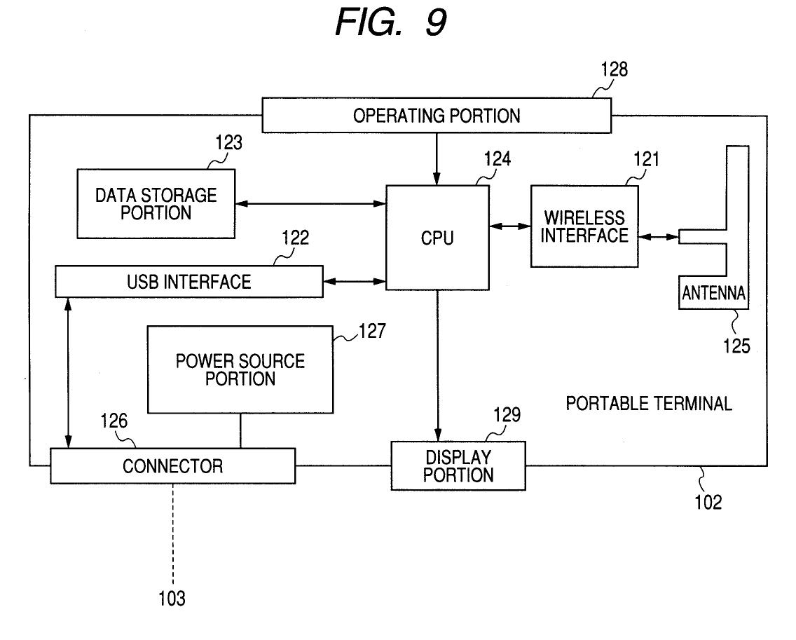

FIG. 9 is a functional block diagram showing an example of the configuration of the portable terminal 102. The data which are received by the wireless interface 121 via an external or internal antenna 125 are processed by a CPU 124, and then stored in the data storage portion 123 configured by a FLASH memory, a ROM, and the like, for a predetermined time period.

The data in the data storage portion 123 are read out by the CPU 124, and then supplied from the USB interface 122 to the USB cable 103 via a connector 126. The portable terminal further comprises: a power source portion 127 configured by a rechargeable battery; an operating portion 128 having operation buttons through which the assistant instructs the CPU 124; and a display portion 129 which displays various information supplied from the CPU 124.

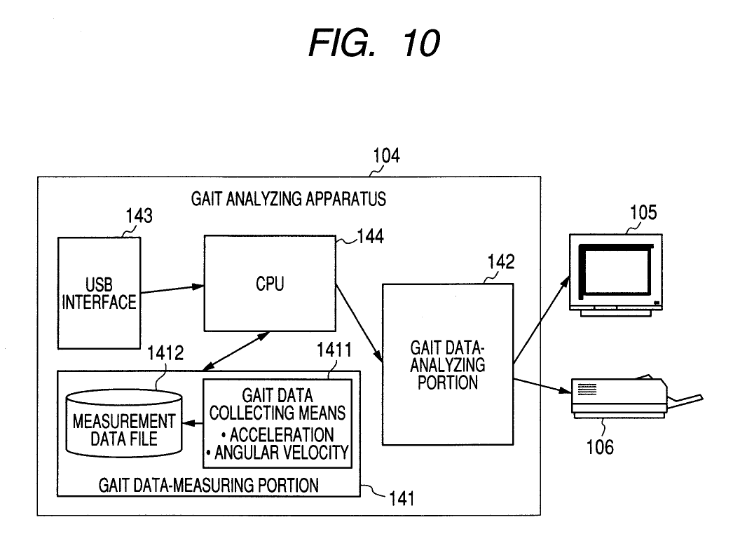

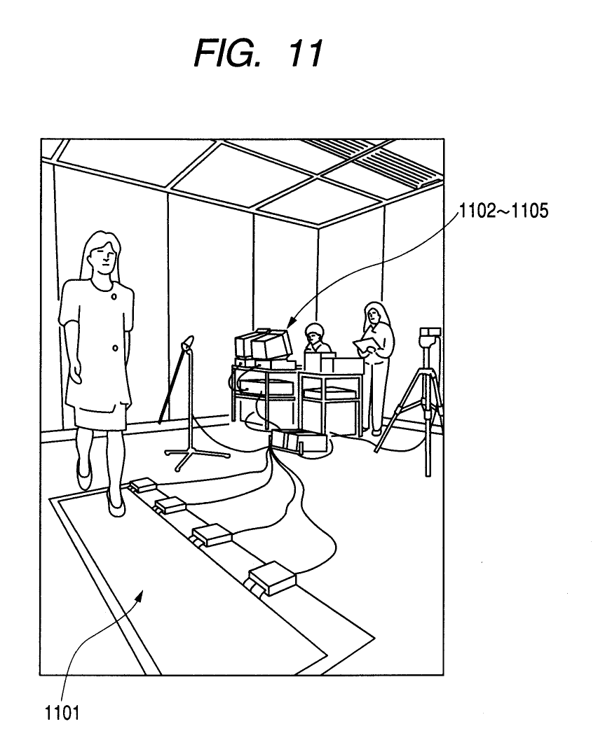

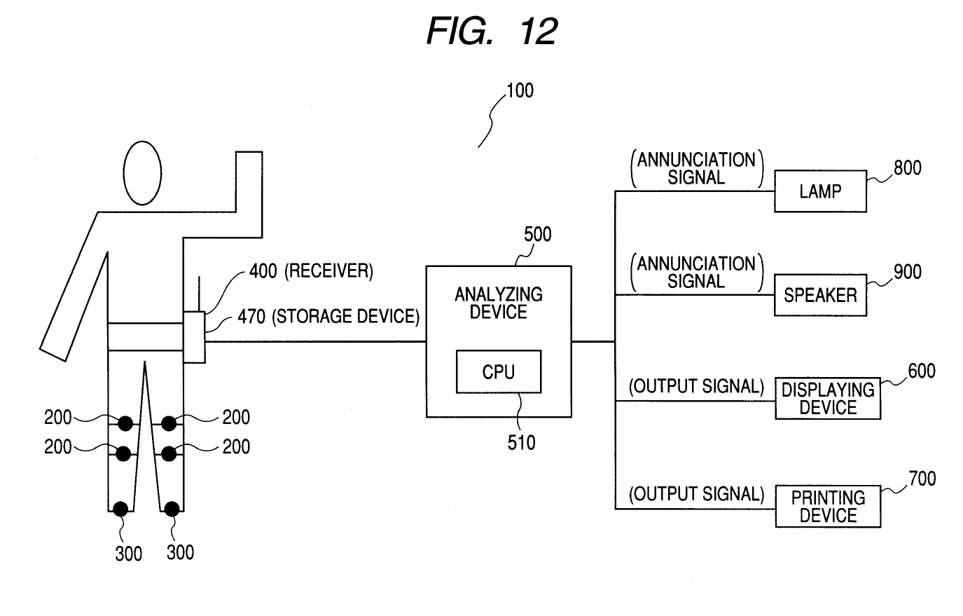

FIG. 10 is a functional block diagram showing an example of the configuration of the gait analyzing apparatus 104. The data from the portable terminal 102 are passed to a CPU 144 via a USB interface 143. Gait data collecting means 1411 of the gait data-measuring portion 141 obtains the gait data from the CPU 144, and stores the data in a measurement data file 1412 for a predetermined time period. The stored measurement data are acceleration data and angular velocity data.

The CPU 144 reads out the stored data from the measurement data file 1412, passes the data to the gait data-analyzing portion 142, and causes the portion to execute various analyzing processes. Results of the analyses are supplied to a displaying device 105 and a printing device 106.

The analysis results can be delivered directly or via the network to an external apparatus or agency, a doctor and physical therapist who are rehabilitation instructors, and the walking person oneself.

Next, the procedure of collecting data from the gait sensor 101 to the gait data-measuring portion 141 via the portable terminal 102 will be described with reference to FIGS. 2, 8, 9.

(1) The walking person M attaches the gait sensor 101 to the foot.
(2) The assistant R carries the portable terminal 102.
(3) The assistant R presses a measurement start button of the operating portion 128 of the portable terminal 102.
(4) When the measurement start button is pressed, the portable terminal 102 transmits a start signal to the gait sensor 101 via the wireless interface 121.
(5) Upon receiving the start signal, the gait sensor 101 converts the measurement values of the acceleration sensors 111 and angular velocity sensors 112 which are on the gait sensor, to digital values by the A/D converter, and transmits the digital values to the portable terminal 102 via the wireless interface 113.
(6) The portable terminal 102 receives the data via the wireless interface 121, and stores the received data into the data storage portion 123.
(7) After elapse of an appropriate time period (for example, after walking of 10 m), the assistant R presses a stop button of the operating portion 128 of the portable terminal 102 to stop the data collection.
(8) When the stop button is pressed, the portable terminal 102 transmits a stop signal to the gait sensor 101 via the wireless interface 121.
(9) Upon receiving the stop signal, the gait sensor 101 stops the data conversion and the data communication. The measurements of (3) to (8) above are repeated plural times.
(10) The portable terminal 102 is connected to the USB interface 143 of the gait analyzing apparatus 104 through the USB cable 103, and, in response to instructions from the operating portion 128, transfers the stored data of the data storage portion 123 to the gait data-measuring portion 141.

The configuration of the gait data-analyzing portion 142, the signal processing procedure of the gait analysis algorithm, and data processing procedure of the gait analysis application are the same as the first embodiment as shown in FIGS. 5 to 7 and thus, the detailed explanation of them will be omitted.

Hereinafter, application examples of the present invention will be described.

(1) At the same time when the gait data are accumulated in the files, the gait analyzing apparatus 104 can calculate in real time and display the locus of the present gait on the screen. Therefore, it is possible to know in real time also the motion of a patient in a hospital.
(2) The connection between the portable terminal 102 and the gait analyzing apparatus 104 can be realized also by means other than the USB cable 103, for example, a wireless LAN.
(3) The data storage portion 123 of the portable terminal 102 is configured by detachable storing means (a portable HDD, a USB clip memory, or the like). The stored data can be passed to the gait analyzing apparatus 104 by detaching the storing means from the terminal and directly connecting the storing means to the apparatus.
(4) The gait sensor 101 comprises a calculating function due to the CPU 15, and primary processes such as the process of filtering the measurement values are executed in the gait sensor 101, whereby the data amount of the wireless communication to the portable terminal 102 can be reduced so that the communication speed can be improved.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A gait analysis system comprising:
a gait sensor which is to be attached to a foot portion of one foot or both feet of a walking person, and which wirelessly outputs detection data of at least one of an acceleration and an angular velocity;
a wireless communication device which receives the detection data; and
a gait analyzing apparatus which, based on the detection data obtained via said wireless communication device, calculates two- or three-dimensional position information and status information of the foot portion at an arbitrary time.

2. A gait analysis system according to claim 1, wherein a start and end of the obtaining of the detection data are determined on the bases of continuation of a suspending state of the foot portion for a constant time period.

3. A gait analysis system according to claim 1, wherein detection of a suspending state of the foot portion is determined by satisfying at least one of conditions that a detection value of the angular velocity is not larger than a certain threshold, and that a detection value of the acceleration is not larger than a certain threshold.

4. A gait analysis system according to claim 1, wherein said gait sensor comprises at least one of an X-Y-Z direction acceleration sensor and an X-Y-Z direction angular velocity sensor.

5. A gait analysis system according to claim 1, wherein said gait sensor is attached to a vicinity of a toe portion of a footwear of the walking person.

6. A gait analysis system according to claim 1, wherein said gait sensor is attached to a vicinity of a heel portion of a footwear of the walking person.

7. A gait analysis system according to claim 1, wherein said wireless communication device is a wireless access point formed on a network.

8. A gait analysis system according to claim 7, wherein said gait analyzing apparatus obtains the detection data from said wireless access point via said network.

9. A gait analysis system according to claim 1, wherein said gait analyzing apparatus calculates a relative moving distance by a gait analysis algorithm which processes the detection data, and, based on the relative moving distance, calculates the status information including at least one of data of a gait position, a gait time period, a stride, a gait speed, a ratio of stance, and a ratio of swing.

10. A gait analysis system according to claim 1, wherein said gait analyzing apparatus integrates at least one of an angular velocity and an acceleration in each step to calculate an angle, velocity, and moving distance which are two- or three-dimensional.

11. A gait analysis system according to claim 1, wherein said gait analyzing apparatus uses at least one of an integration error of the angular velocity, and an integration error of the acceleration in order to correct a distance error of each step.

12. A gait analysis system according to claim 1, wherein said gait analyzing apparatus calculates the position information and the status information in real time with respect to the detection data.

13. A gait analysis system according to claim 1, wherein said gait analyzing apparatus does not transmit the detection data of a predetermined time period to said wireless communication device, and holds the detection data in a memory resource of said gait analyzing apparatus.

14. A gait analysis system according to claim 1, wherein said gait analyzing apparatus communicates with said gait sensor via said wireless communication device to perform tuning on said gait sensor.

15. A gait analysis system comprising:
a gait sensor which is to be attached to a foot portion of one foot or both feet of a walking person, and which wirelessly outputs detection data of at least one of an acceleration and an angular velocity;
a portable terminal which receives the detection data, and which stores the data for a predetermined time period; and
a gait analyzing apparatus which, based on the detection data obtained from said portable terminal, calculates twos or three-dimensional position information and status information of the toot portion at an arbitrary time.

16. A gait analysis system according to claim 15, wherein said gait sensor comprises at least one of an X-Y-Z direction acceleration sensor and an X-Y-Z direction angular velocity sensor.

17. A gait analysis system according to claim 15, wherein said gait sensor is attached to a vicinity of a toe portion of a footwear of the walking person.

18. A gait analysis system according to claim 15, wherein said gait sensor is attached to a vicinity of a heel portion of a footwear of the walking person.

19. A gait analysis system according to claim 15, wherein said portable terminal is carried by an assistant who is in a vicinity of the walking person.

20. A gait analysis system according to claim 15, wherein said gait analyzing apparatus obtains the detection data from said portable terminal via a USB interface.

21. A gait analysis system according to claim 15, wherein said gait analyzing apparatus obtains the detection data from said portable terminal via a detachable storing section.

22. A gait analysis system according to claim 15, wherein said gait analyzing apparatus calculates a relative moving distance by a gait analysis algorithm which processes the detection data, and, based on the relative moving distance, calculates status information including at least one of data of a gait position, a gait time period, a stride, a gait speed, a ratio of stance, and a ratio of swing.

23. A gait analysis system according to claim 15, wherein said gait analyzing apparatus integrates at least one of an angular velocity and an acceleration in each step to calculate an angle, velocity, and moving distance which are two- or three-dimensional.

24. A gait analysis system according to claim 15, wherein said gait analyzing apparatus delivers output information to at least one of a doctor, a physical therapist, and the walking person, directly or via a network.
